(19) United States
(12) Patent Application Publication
(10) Pub. No.: US 2005/0246018 A1
(43) Pub. Date: Nov. 3, 2005

(54) Injectable accommodation composition

(75) Inventors: Robert H. Grubbs, South Pasadena, CA (US); Axel Brait, San Rafael, CA (US); Shiao H. Chang, Pasadena, CA (US); Okihiro Nishi, Osaka (JP); Daniel M. Schwartz, San Francisco, CA (US)

Correspondence Address: FULBRIGHT & JAWORSKI, LLP, 1301 MCKINNEY, SUITE 5100, HOUSTON, TX, 77010-3095, US

(73) Assignee: CALHOUN VISION, INC., Pasadena, CA (US)

(21) Appl. No.: 11/118,253

(22) Filed: Apr. 29, 2005

Related U.S. Application Data
(60) Provisional application No. 60/567,331, filed on Apr. 30, 2004.

(51) Int. Cl. A61F 2/16

(52) U.S. Cl. 623/6.37; 623/6.56

(57) ABSTRACT

The invention relates to a novel composition for improving the accommodation capability of an intraocular lens (IOL). In one embodiment, the composition can be injected into the capsular bag where it surrounds an implanted IOL anchoring the IOL to the capsular bag. Alternatively, for anterior chamber IOLs, the composition can be injected into the bag behind the lens. The material has a refractive index similar to that of aqueous thereby reducing any potential interference with the implanted IOL. Accommodation is provided by the mixture of the crosslinked composition caused by the flexing of the muscles. The novel composition is particularly useful in enhancing accommodation for adjustable intraocular lenses.

### CROSS-REFERENCE TO RELATED APPLICATIONS

This application claims priority to U.S. Provisional application Ser. No. 60/567,331 filed Apr. 30, 2004.

The invention relates to a novel composition for improving the accommodation capability of an intraocular lens (IOL). In one embodiment, the composition can be injected into the capsular bag where it surrounds an implanted IOL anchoring the IOL to the capsular bag. Alternatively, the IOL can be captured by the anterior capsule, the composition can be injected into the bag behind the lens. The material has a refractive index similar to that of aqueous humor thereby reducing any potential interference with the implanted IOL. Accommodation is provided by the mixture of the crosslinked composition caused by the flexing of the muscles. The novel composition is particularly useful in enhancing accommodation for adjustable intraocular lenses.

### BACKGROUND OF THE INVENTION

Cataract extraction is the most common ophthalmic surgical procedure performed in the United States. Extracapsular cataract extraction involves cutting a portion of the anterior capsule (anterior capsulorhexis) followed by removal of the nucleus. Alternatively, a probe may be inserted through the anterior capsule and ultrasonically vibrated, transforming lens material into an emulsion, which is then irrigated and aspirated from the capsular bag (phacoemulsification). After removal of the natural lens, images no longer focus on the retina and a replacement lens must be provided for clear vision. Replacement lenses can be glasses, contact lenses or intraocular lenses. Of these, intraocular lenses give the greatest convenience and undistorted vision, however, lack the ability of a natural lens to accommodatively focus on near and far objects.

When a person looks at an object, light is reflected from the object through the cornea, the aqueous humor, through the pupil and into the lens which converges the light through the vitreous body onto the retina. To clearly focus on near objects, light rays must be bend more. To accomplish this, the lens becomes more curved and thicker. Most of this change comes from pulling and relaxing the capsular bag at its equator. The equator of the bag is attached to the ciliary muscle by filaments called the zonules which are in turn attached to the ciliary muscle. When looking at a near object, the ciliary muscle tenses and contracts moving the muscle slightly inward and relaxing the pull on the zonules, allowing the capsular bag to become more curved and thickened from front to back. The lens itself is composed of interlocking fibers which affect the elastic movement of the lens so that as the lens changes shape the fibers alter their curvature. As a person ages, the accommodative ability of the lens decreases due to changes in the eye. Age related eye changes include thickening and hardening of the lens, an increase in the amount of insoluble protein in the lens, a migration in the points of attachment of the zonules away from the equator of the capsule, and partial liquefaction of the vitreous body.

Several attempts have been made to provide the eye with focal length accommodation. The most familiar of these are bi or multi-focal lenses. These are used in glasses, contacts, and intraocular lenses but have a disadvantage in that the focal accommodation is dependent upon direction of focus. These lenses do not provide true accommodation. The accommodating implant provides vision over a continuous range of distance by affecting a change in the vergence power of the eye resulting from the implant design that changes eye optical power or implant position in response to a stimulus.

U.S. Pat. No. 4,254,509 discloses a lens which takes advantage of the ciliary muscle. However, this lens is placed in the anterior chamber of the eye. Such implants are at times accompanied by complications such as damage to the vascular iris.

U.S. Pat. No. 4,253,199 discloses a lens attached directly to the ciliary body. The lens is in a more natural position but requires suturing to the ciliary body risking massive rupture during surgery and bleeding from the sutures.

U.S. Pat. No. 4,685,922, incorporated herein by reference, discloses a chambered lens system for which the refractive power can be changed. Such alteration is permanent, accomplished by rupture of the chambers.

U.S. Pat. No. 4,790,847 provides a single lens for in capsular bag implantation using rearwardly biased haptics which engage the capsular bag at its equator and move the lens forward and backward upon contraction and relaxation of the ciliary muscles.

U.S. Pat. No. 4,842,601, incorporated herein by reference, discloses a two section deformable lens assembly for implanting in the capsular bag. The lens allows division of refractive power and takes advantage of the action of the ciliary body and zonules on the capsular bag. This lens system is assembled after insertion.

U.S. Pat. No. 4,892,543, discloses another two lens assembly for placement in the posterior chamber, possibly in the bag where the capsular bag is not removed. This lens allows dividing the refractive power between two lenses and introduces a variable focal length in one of the lenses by compressing a flexible wall of one lens against the convex surface of the second fixed lens. This requires that the first and second lens be in substantially adjacent positions.

U.S. Pat. No. 4,932,966, incorporated herein by reference, presents an accommodative lens in which two lenses joined at their periphery enclosed a fluid filled sack, accommodation being accomplished selectively changing the fluid pressure in the sac. One lens is a rigid base lens and the other lens is membrane-like, the equatorial diameter of the lens assembly being substantially that of a dilated pupil and is supported by bladders or haptics.

U.S. Pat. No. 5,275,623 discloses dual and thick lens optics capable of accommodating focus at a range of distances in a unitary structure. It uses the eye capsule's natural shaping from the ciliary body to accommodate the focus.

PCT Application No. WO 60/61036 discloses an open chamber, elliptical, accommodating lens system. It uses a pair of lenses attached to each other by to or more haptics. The system uses the eye capsule's natural shaping from the ciliary body to accommodate the focus of the lenses.

### BRIEF SUMMARY OF THE INVENTION

The present invention provides an injectable composition which, when crosslinked in vivo, allows an implanted intraocular lens (IOL) to provide accommodating focus at a range of distances. In one embodiment, the composition binds to the walls of the capsular bag and anchors the IOL to the bag aiding the accommodation process. Alternatively, as in the case of anterior capsule captured IOLs, the composition is placed behind the lens. After the composition is crosslinked in vivo, it exhibits sufficient elastic properties such that as the muscles on either side of the capsular bag extend or contract, they cause the composition to extend or contract. This, in turn, shifts the position of the IOL forward or backward, thus providing accommodation with change of focus. In addition, the curvature of the filled capsular bag changes with the muscle movement during accommodation. The elastic and mechanical properties of the composition can be adjusted, in vivo, through the use of macromers present in the composition, exposure to an external stimulus such as light.

The injectable composition comprises a first and second component which, when combined in the eye, form a polymer matrix within the capsular bag that surrounds an IOL implanted in the eye. Dispersed within the matrix is a macromer or mixture of macromers capable of stimulus induced polymerization.

The matrix then serves to assist in the accommodation process. As the zonules pull at the capsular bag, the shape of the bag changes. This in turn puts pressure on the polymer matrix causing the matrix to change shape and thereby shift the position of the IOL. The macromer present in the matrix can be used to adjust the physical properties of the matrix making it more or less flexible. This in turn, affects that movement of the IOL when the capsular bag changes shape. Both the shape change of the capsular bag and the movement of the IOL as a result of the change of this composition provide the accommodation in present invention.

### BRIEF DESCRIPTION OF THE DRAWINGS

For a more complete understanding of the present invention, reference is now made to the following descriptions taken in conjunction with the accompanying drawing, in which:

FIG. 1 is a vertical sectional view of an eye;

FIG. 2 is a cross-section of a capsular bag with an IOL and the composition of the invention in place.

FIGS. 3A and 3B are of photographs of excised pig capsular bags containing the accommodation composition of the invention.

FIG. 4A shows a view of an excised crosslinked accommodation composition from a pig's capsular bag.

FIG. 4B shows the optical properties of the accommodation composition of FIG. 4A.

FIGS. 5A and 5B show views of a refilled capsular bag from a rabbit's eye.

FIG. 6 shows the optical properties of a crosslinked accommodation composition from a rabbit's eye.

### DETAILED DESCRIPTION OF THE INVENTION

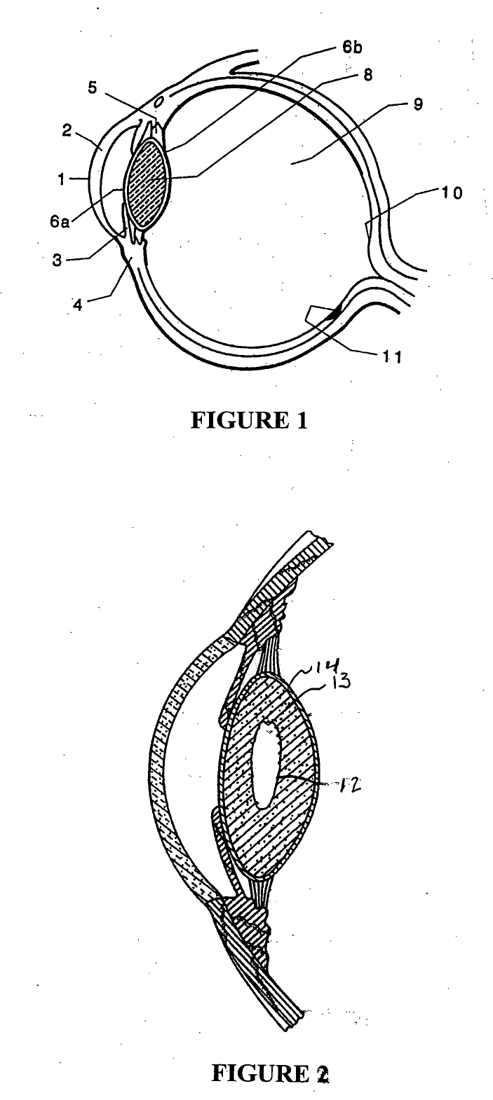

FIG. 1 shows a cross-section of the eye. As light enters the eye, it passes through the cornea 1; through the aqueous humor in the anterior chamber 2; through the pupil located centric of iris 3; through the anterior wall of the capsular bag 6a; is convergently refracted by the lens 8; passes through the posterior wall of the capsular bag 6b; through the vitreous humor 9, to the retina 10 at the fovea 11. The shape of the lens capsule is controlled by ciliary muscle 4 attached to the capsule by filaments called zonules 5.

When the natural lens 8 is removed through known processes such as phacoemulsification, an intraocular lens such as that shown in FIG. 2 can restore focusing. The IOL 12 can be a standard lens whose optical properties are pre-set prior to implantation or an adjustable lens whose properties can be manipulated post implantation. An adjustable lens useful in the practice of this invention is described in U.S. Pat. No. 6,450,642.

The IOL 12 is secured in place through the use of haptics which engage the walls of the capsular bag 14. The haptics can be of any conventional design. The lens can be placed anterior or interior to the capsular bag. In either case, the lens is anchored using standard techniques.

Once the IOL 12 is in place, the capsular bag 14 is then filled with the composition of the invention. When the composition cures, it provides an anchor for the IOL 12 to the capsular bag 14 and helps in the accommodation process. When the lens used is an anterior capsule captured IOL (AC-IOL), the lens may also acts as a plug to hold the composition in the bag whilst the curing step is completed.

In one embodiment, the composition also comprises macromers possessing functional groups. The physical properties of the cured composition can be adopted by cross-linking the functionalized macromer. This is accomplished by exposing the functionalized macromers to an external stimulus such a light. In a preferred embodiment, the external stimulus is ultraviolet light.

The composition used in the practice of the invention should exhibit a viscosity similar to that of the natural lens, typically between 100 and 1000 Pa and should have an elastic modulus similar to that of the natural lens material. For example for a young human lens, an elastic modulus of from 400 to 600 Pa is preferred. This allows the composition to deform and recoil when the muscles exert and release force on the zonules attached to the capsular bag. The composition should initially also be of sufficiently low viscosity to allow injection into the capsular bag.

The composition should also have optical properties that do not interfere with the function of the IOL. In general, this means that the refraction index of the material in the capsular bag should be similar to that of a lens or aqueous hymen. Typically, this would be about 1.41-1.43.

The composition may comprise a fully crosslinked polymer that can be directly injected into the capsular bag or it may comprise one or more precursors, which, when injected into the capsular bag, cure to form a crosslinked structure. The latter materials can include crosslinkable esters of hyaluronic acid, collagen, hydrogels of poly (N,N.-isopropylacrylamide and functional silicone compounds. Examples of collagen based materials useful in the practice of the invention include those disclosed in U.S. Pat. Nos. 5,476,515 and 5,910,537.

Illustrative examples of a suitable first polymer matrix include: poly-acrylates such as poly-alkyl acrylates and poly-hydroxyalkyl acrylates; poly-methacrylates such as poly-methyl methacrylate (“PMMA”), poly-hydroxyethyl methacrylate (“PHEMA”), and poly-hydroxypropyl methacrylate (“PHPMA”); poly-vinyls such as poly-styrene and poly-N-vinylpyrrolidone (“PNVP”); poly-siloxanes such as poly-dimethylsiloxane, dimethylsiloxane diphenylsiloxane copolymers, dimethylsiloxane methylphenylsiloxane copolymers; poly-phosphazenes; urethanes and copolymers thereof. U.S. Pat. No. 4,260,725 and patents and references cited therein (which are all incorporated herein by reference) provide more specific examples of suitable polymers that may be used to form the first polymer matrix.

In preferred embodiments, the first polymer matrix generally possesses a relatively low glass transition temperature (“T_{g}”) such that the resulting IOL tends to exhibit fluid-like and/or elastomeric behavior, and is typically formed by crosslinking one or more polymeric starting material wherein each polymeric starting material includes at least one crosslinkable group. Illustrative examples of suitable crosslinkable groups include but are not limited to hydride, vinyl, acetoxy, alkoxy, amino, anhydride, aryloxy, carboxy, enoxy, epoxy, halide, isocyano, olefinic, and oxime. In more preferred embodiments, each polymeric starting material includes terminal monomers (also referred to as endcaps) that are either the same or different from the one or more monomers that comprise the polymeric starting material but include at least one crosslinkable group. Consequently, other embodiments include crosslinkers that have reactive groups attached as side-groups along the backbone and/or terminal endcaps. In other words, the terminal monomers begin and end the polymeric starting material and include at least one crosslinkable group as part of its structure. Although it is not necessary for the practice of the present invention, the mechanism for crosslinking the polymeric starting material preferably is different than the mechanism for the stimulus-induced polymerization of the components that comprise the refraction modulating composition. For example, if the refraction modulating composition is polymerized by photo-induced polymerization, then it is preferred that the polymeric starting materials have crosslinkable groups that are polymerized by any mechanism other than photo-induced polymerization.

An especially preferred class of polymeric starting materials for the formation of the first polymer matrix is poly-siloxanes (also know as “silicones”) endcapped with a terminal monomer which includes a crosslinkable group selected from the group comprising acetoxy, amino, alkoxy, halide, hydroxy, vinyl, hydride and mercapto. Because silicone IOLs tend to be flexible and foldable, generally smaller incisions may be used during the IOL implantation procedure. An example of an especially preferred polymeric starting material is bis(diacetoxymethylsilyl)-polydimethylsiloxane (which is poly-dimethylsiloxane that is endcapped with a diacetoxymethylsilyl terminal monomer). Another example involves hydrosilylation reactions between the vinyl- and the hydride-functionalized silicones in presence of a catalyst, preferably a platinum complex and is similar to the compositions described in the U.S. Pat. No. 5,411,553 and others.

In the present invention, the first polymer matrix is formed in vivo. This is accomplished in injecting the precursors for the first polymer matrix as well as the refraction-and/or shape-modifying composition into a body cavity and allowing the precursors of the first polymer matrix to cure in the presence of the refraction- and/or shape-modifying composition. The curing is accomplished through catalytic polymerization of the first and second precursor.

Where the first polymer matrix is a silicone-based matrix, two types of precursors are required to form the first polymer matrix useful in the practice of the invention. The first precursor comprises one or more vinyl-containing polyorganosiloxanes and the second precursors comprise one or more organosilicon compounds having silicon-bonded hydride groups which react with the vinyl groups of the first precursor.

The first precursor preferably has an average of at least two silicone-bonded vinyl groups per molecule. The number of vinyl groups can vary from two per molecule. For example the first precursor can be a blend of two or more polyorganosiloxanes in which some of the molecules have more than two vinyl groups per molecule and some have less than two vinyl groups per molecule. Although it is not required that the silicon-bonded vinyl groups be located in the alpha, omega (i.e. terminal) positions, it is preferred that at least some of the vinyl radicals be located at these positions. The vinyl groups are located at the polymer ends because such polyorganosiloxanes are economical to produce and provide satisfactory products. However, because of the polymeric nature of the first precursor, its preparation may result in products that have some variation in structure, and some vinyl groups may not be in the terminal position, even if the intent is to have them in these positions. Thus, the resulting polyorganosiloxanes may have a portion of the vinyl radicals located at branch sites.

The polyorganosiloxanes of the first precursor are preferably essentially linear polymers that may have some branching. The polyorganosiloxanes may have silicon-oxygen-silicon backbones with an average of greater than two organo groups per silicon atom. Preferably, the first precursor is made up of diorganosiloxane units with triorganosiloxane units for endgroups, but small amounts of monoorganosiloxane units and SiO₂ may also be present. The organo groups preferably have less than about 10 carbon atoms per group and are each independently selected from monovalent hydrocarbon groups such as methyl, ethyl, vinyl propyl, hexyl and phenyl and monovalent substituted hydrocarbon groups such as perfluoroalkylethyl groups . Examples of first precursors include dimethylvinylsiloxy endblocked polydimethylsiloxane, methylphenylvinylsiloxy endblocked polydimethylsiloxane, dimethylvinylsiloxy endblocked polymethyl-(3,3,3-triflouropropyl) siloxane, dimethylsiloxy endblocked polydiorganosiloxane copolymers of dimethylsiloxane units and methylphenylsiloxane units and methylphenylvinylsiloxy endblocked polydiorganosiloxane copolymers of dimethylsiloxane units and diphenylsiloxane units and the like. The polydiorganosiloxane can have siloxane units such as dimethylsiloxane units, methylphenylsiloxane units, methyl-(3,3,3-trifluoropropyl)siloxane units, monomethylsiloxane units, monophenylsiloxane units, dimethylvinylsiloxane units, trimethylsiloxane units, and SiO₂ units. Polyorganosiloxanes of the first precursor can be single polymers or mixtures of polymers. These polymers may have at least fifty percent of the organic groups as methyl groups. Many polyorganosiloxanes useful as the first precursor are known in the art and are commercially available. A preferred first precursor is polydimethylsiloxane endblocked with dimethylvinylsiloxy units or methylphenylsiloxy units having a viscosity of from about 500 to 100,000 centipoise at 25° C.

The second precursor includes organosilicon compounds containing at least 2, and preferably at least 3, silicon-bonded hydride groups, i.e., hydrogen atoms, per molecule. Each of the silicon-bonded hydride groups is preferably bonded to a different silicon atom. The remaining valences of the silicon atom are satisfied by divalent oxygen atoms or by monovalent groups, such as alkyl having from 1 to about 6 carbon atoms per group, for example methyl, ethyl, propyl, isopropyl, butyl, tertiary butyl, pentyl hexyl, cyclohexyl, substituted alkyl groups, aryl groups, substituted aryl groups and the like. The silicon-bonded hydride group containing organosilicon compounds can be homopolymers, copolymers and mixtures thereof which contain siloxane units of the following types: RSiO_{1.5}, R₂SiO, RHSiO, HsiO_{1.5}, R₂Hsi_{0.5}, H₂SiO RH₂ SiO^{0.5}, and SiO where R is the monovalent group, for example, as defined above. Examples include polymethylhydrogensiloxane cyclics, copolymers of trimethylsiloxy and methylhydrogensiloxane, copolymers of dimethylsiloxy and methylhydrogensiloxane, copolymers of trimethylsiloxy, dimethylsiloxane and methylhydrogensiloxane, copolymers of dimethylhydrogensiloxane, dimethylsiloxane and methylhydrogensiloxane and the like. Also needed is a crosslinker resin. This resin is a multifunctional vinyl silicone of certain molecular weight, branched structure and functionality. The other crosslinker is the multifunctional silicone hydride of certain molecular weight, branched structure and functionality.

The platinum group metal catalyst component can be any of the compatible platinum group metal-containing catalysis known to catalyze the addition of silicone-bonded hydrogen atoms (hydride groups) to silicon-bonded vinyl radicals. Platinum group metal-containing catalysts can be any of the known forms which are compatible, such as platinic chloride, salts of platinum, chloroplatinic acid and various complexes. The platinum group metal-containing catalyst can be used in any catalytic quantity, such as in an amount sufficient to provide at least about 0.1 ppm weight of platinum group metal (calculated as elemental metal) based on the combined weight of the first and second precursors. Preferably, at least 10 ppm, or more preferably, at least about 20-40 ppm by weight of platinum group metal based on the combined weight of the first and second precursors is used.

The first component further comprises a catalyst to induce the polymerization of the first and second components to form the polymer matrix in the capsular bag.

The composition of the invention may also comprise a modifying composition or macromer which is capable of modifying the characteristics of the composition in vivo. In the preferred embodiment, the macomers are capable of modifying the elastomer properties of this polymer matrix. This is accomplished by stimulus induced polymerization of the macromer, which is further accomplished through the use of functional groups on the macromers that are capable of stimulus induced polymerization. Upon exposure to the appropriate stimulus, the macromer polymerizes to form a second polymer matrix. This polymerization causes changes in the properties of the crosslinked composition.

The modifying composition that is used in practice of the invention is as described above except that it has the preferred requirement of biocompatibility. The refraction-and/or shape-modifying composition is capable of stimulus-induced polymerization and may be a single component or multiple components so long as: (i) it is compatible with the formation of the first polymer matrix; (ii) it remains capable of stimulus-induced polymerization after the formation of the first polymer matrix; (iii) it is freely diffusible within the first polymer matrix. In general, the same type of monomer that is used to form the first polymer matrix may be used as a component of the shape-modifying composition. The monomers will often contain functional groups that are capable of stimulus-induced polymerization. However, because of the requirement that the modifying monomers must be diffusable within the first polymer matrix, the modifying monomers generally tend to be smaller (i.e., have lower molecular weights) than the first polymer matrix network, i.e., the diffusible materials have to be of molecular weight less than for instance the molecular weight between crosslinks of the first polymer matrix. In addition to the one or more monomers, the composition may include other components such as initiators and sensitizers that facilitate the formation of the second polymer matrix. In addition, to provide the UV-blocking properties similar to the natural eye, UV-absorbers may also be incorporated as a component of the refraction- and/or shape-modifying composition.

In preferred embodiments, the stimulus-induced polymerization is photopolymerization. In other words, for the one or more monomers that comprise the refraction- and/or shape modulating composition, each preferably includes at least one functional group that is capable of photopolymerization. Illustrative examples of such photopolymerizable groups include but are not limited to acrylate, allyloxy, cinnamoyl, methacrylate, stibenyl, and vinyl. In more preferred embodiments, the refraction- and/or shape-modifying composition includes a photoinitiator (any compound used to generate free radicals) either alone or in the presence of a sensitizer and UV-absorbers. Examples of suitable photoinitiators include acetophenones (e.g., substituted haloacetophenone, and diethoxyacetophenone); 2,4-dichloromethyl-1,3,5-triazines; benzoin methyl ether; and o-benzoly oximino ketone and silicone derivatives thereof. Examples of suitable sensitizers include p-(dialkylamino)aryl aldehyde; N-alkylindolylidene; and bis[p-(dialkylamino)benzylidiene] ketone and silicone derivatives thereof. Examples of UV-absorbers include but are not limited to the benzophenones and their derivatives, benzotriazoles and their derivatives, and others that are known in the art of UV-blocking materials.

One class of macromers useful in the practice of the invention includes poly-siloxanes endcapped with a terminal siloxane moiety that includes a photopolymerizable group. An illustrative representation of such a monomer is:
X-Y-X¹
wherein Y is a siloxane which may be a monomer, a homopolymer or a copolymer formed from any number of siloxane units, and X and X1 may be the same or different and are each independently a terminal siloxane moiety that includes a photopolymerizable group. An illustrative example of Y includes: 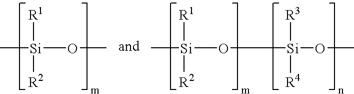
wherein: m and n are independently each an integer and R¹, R², R³, and R⁴ are independently each hydrogen, alkyl (primary, secondary, tertiary, cyclo), aryl, or heteroaryl. In preferred embodiments, R¹, R², R³, and R⁴ are C₁-C₁₀ alkyl or phenyl. Because shape-modifying composition monomers with a relatively high aryl content have been found to produce larger changes in the refractive index of the inventive lens, it is generally preferred that at least one of R¹, R², R³, and R⁴ is an aryl, particularly phenyl. In more preferred embodiments, R¹, R², and R³ are the same and are methyl, ethyl or proply and R⁴ is phenyl.

Illustrative examples of X and X1 (or X1 and X depending on how the RSMC polymer is depicted) are 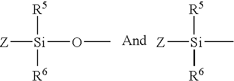
respectively wherein:

R⁵ and R⁶ are independently each hydrogen, alkyl, aryl, or heteroaryl; and Z is a photopolymerizable group.

In preferred embodiments, R⁵ and R⁶ are independently each a C₁-C₁₀ alkyl or phenyl and Z is a photopolymerizable group that includes a moiety selected from the group consisting of acrylate, allyloxy, cinnamoyl, methacrylate, stibenyl, and vinyl. In more preferred embodiments, R⁵ and R⁶ is methyl, ethyl, or propyl and Z is a photopolymerizable group that includes an acrylate or methacrylate moiety.

In especially preferred embodiments, the refraction- and/or shape-modifying composition monomer is of the following formula: 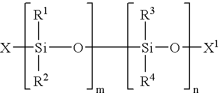
wherein X and X¹ are the same and R¹, R², R³, and R⁴ are as defined previously. Illustrative examples of such shape-modifying composition monomers include dimethylsiloxane-diphenylsiloxane copolymer endcapped with a vinyl dimethylsilane group; dimethylsiloxane-methylphenylsiloxane copolymer endcapped with a methacryloxypropyl dimethylsilane group; and dimethylsiloxane endcapped with a methacryloxypropyldimethylsilane group. Although any suitable method may be used, a ring-opening reaction of one or more cyclic siloxanes in the presence of triflic acid has been found to be a particularly efficient method of making one class of inventive shape-modifying composition monomers. Briefly, the method comprises contacting a cyclic siloxane with a compound of the formula: 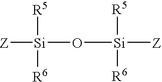
in the presence of triflic acid wherein R⁵, R⁶, and Z are as defined previously. The cyclic siloxane may be a cyclic siloxane monomer, homopolymer, or copolymer. Alternatively, more than one cyclic siloxane may be used. For example, a cyclic dimethylsiloxane tetramer and a cyclic methyl-phenylsiloxane trimer/tetramer are contacted with bis-methacryloxypropyltetramethyldisiloxane in the presence of triflic acid to form a dimethyl-siloxane methyl-phenylsiloxane copolymer that is endcapped with a methacryloxypropyl-dimethylsilane group, an especially preferred shape-modifying composition monomer. The accommodation composition also may crosslink these.

The IOL that may be used in the practice of the invention include all types of prefabricated IOLs including single lens IOL, adjustable IOLs, multi lens IOLs, and accommodating IOLs such as those described in U.S. Pat. No. 5,275,623. In the case of the latter both type of lenses, the composition of the invention may be used to fill the space between the different lenses as well as any space between the lens and the capsular bag. In the case of lenses captured by the anterior capsule, the composition of the invention fills the entire capsular bag. The lens acts as a plug, holding the composition in the capsular bag during curing.

In practice of the invention, the existing lens is removed from the patient's eye by any standard procedure, preferably phacoemulsification. An intraocular lens is then implanted, using standard surgical procedures. Once the lens has been properly positioned, the composition of the invention is then introduced into the capsular bag.

In one embodiment accomplished by injecting the composition into the capsular bag, filling the space between the bag and the lens, as in the case of a null or two lens system. The accommodation composition fills the space between the individual lenses as well as between the lenses and the capsular bag. The AC-IOL can be as a plug used to seal the capsularhexus of the capsular bag.

In the case of a multiple component system, the different components are kept separate until the materials are implanted in the bag with curing taking place in vivo. This is best accomplished with a multichamber syringe such that the components are combined just before the composition is injected into the lens. For lenses placed in front of the capsule by capturing technique, the capsular bag is filled with the composition of the invention while the material is curing. The lens is inserted and secured in the anterior opening of the capsular bag. The lens also acts as a plug to hold the composition in place.

In the practice of the invention, the natural lens is removed by phaco-emulsification leaving the lens capsule intact except for the flap necessary to insert the phaco tip. The monomers or polymer precursors necessary to form the first polymer matrix as well as the refraction or shape-modifying composition are mixed, precured and injected into the body cavity such that the first polymer matrix is formed in the body cavity. Alternately, the first polymer precursor and the refraction- and/or shape modifying composition are mixed, degassed, transferred to a syringe, and cooled to a temperature (between −10° to 0° C.) at which the first polymer matrix crosslinking is inhibited. The shape-modifying composition monomers as well as any initiators required to form the second polymer matrix and other components, such as UV absorber, are mixed with the first polymer matrix monomers before injection into the body cavity.

Prior to the implantation of the accommodating composition into the capsular bag, it may be necessary to irrigate the bag to reduce the possibility of posterior capsular opacification (“PCO”). Proper sealing of the capsular bag may also prevent PCO. Methods for accomplishing irrigation and sealing of the capsular bag are known in the art. For example, POGs may be used to peel the bag.

For the composition of the invention, the curing temperature for the first polymer matrix is the physiological temperature of the eye, for example, in humans in the range of about 35° C. to about 37° C. Lack of mobility of the injected composition preferably occurs about 20 minutes after injection, more preferably within about 10 minutes. Final cure preferably occurs within about 6 hours, more preferably within about 2 hours of injection.

In one embodiment of the invention, the first and second precursors are separated into two discrete compositions. The first composition comprises the first precursor combined with the refraction- and/or shape-modifying composition (macromer), photoinitiator and, where desired, an UV-absorber. In the second composition, the second precursor and catalyst are combined. Alternatively, the catalyst can be combined with the first precursor and the other components combined with the second precursor. The key is to keep the first and second precursors and the catalyst separate until just before the materials are injected into the body cavity.

A preferred way to prepare the accommodation composition of the present invention is through use of a multichamber syringe which keeps the individual components separate until just before the components are injected into the body cavity. While each component may be injected separately, some components may be combined provided that they do not interact such that they fail to perform as required once they are injected into the body cavity. For example, where the first polymer matrix is formed from two separate monomers in the presence of a catalyst, one chamber of the syringe will contain the first monomer and the second chamber will contain the other monomer. The catalyst can be combined with either monomer unless the catalyst will cause the monomer to polymerize in the chamber. Additional components can be combined in one of the other chambers. For example, the refraction- and/or shape-modifying components can be placed in either chamber as well as any other additives. In the case of intraocular lenses, the additives can include UV absorber such as benzotriazoles, benzophenones, phenylesters, cinnamic acid and derivatives and nickel-containing compounds. The additions may also include stimulus induced initiators for crosslinking the macroners in vivo. These are typically photoinitiators with UV based photoinitiators preferred.

A key advantage of the present invention is that properties of the accommodation composition may be modified after implantation within the body. For example, the flexural modulus of the composition may be modified in a post-surgical outpatient procedure.

In addition to the change in the elastomeric properties of the composition, the shape of the resulting polymer matrix can be adjusted. As a result, both mechanisms may be exploited to provide accommodation. In general, the process for modifying the accommodation composition of the invention comprises:
(a) exposing at least a portion of the composition to an external stimulus whereby the stimulus induces the polymerization of the modifying composition. If after formation of the composition and wound healing, no composition property needs to be modified, then the exposed portion is the entire implant. The exposure of the entire composition will lock in the then-existing properties of the implanted implant. However, if an implant characteristic such as the power of an IOL needs to be modified, then only a portion of the implant (something less than the entire implant) would be exposed. In one embodiment, the method of implementing the inventive implant further comprises:
(b) waiting an interval of time; and
(c) re-exposing the portion of the implant to the stimulus.

This procedure generally will induce the further polymerization of the refraction modulating composition within the exposed implant portion. Steps (b) and (c) may be repeated any number of times until the implant has reached the desired implant characteristic. At this point, the method may further include the step of exposing the entire implant to the stimulus to lock-in the desired lens property.

In another embodiment wherein a lens property needs to be modified, a method for implementing an inventive IOL comprises:
(a) exposing a first portion of the lens to a stimulus whereby the stimulus induces the polymerization of the refraction modulating composition; and
(b) exposing a second portion of the lens to the stimulus.

The first lens portion and the second lens portion represent different regions of the lens although they may overlap. Optionally, the method may include an interval of time between the exposures of the first lens portion and the second lens portion. In addition, the method may further comprise re-exposing the first lens portion and/or the second lens portion any number of times (with or without an interval of time between exposures) or may further comprise exposing additional portions of the lens (e.g., a third lens portion, a fourth lens portion, etc.). Once the desired property has been reached, then the method may further include the step of exposing the entire lens to the stimulus to lock-in the desired lens property.

In a third embodiment, the properties of both the lens and the accommodation composition can be manipulated in the manner described above.

### EXAMPLES

A series of experiments were conducted with pig and rabbit eyes using gel compositions both with and without modifying macroner.

### I. Pig Eyes

In these experiments, a series of six pig cadaver eyes were used. The lenses were removed using phacoemulsification followed by capsulorhexis with a diameter of approximately 5 mm. The capsular bag and anterior chamber was then filled with a blend of Part A (Gel 8150, Lot 27930, Nusil Technologies) and Part B (Gel 8150, Lot 27930, Nusil Technologies). A light adaptable Anterior Capsule Captured Intraocular Lens (AC-IOL) was then inserted using a mechanical folding/inserting forceps and placed into the capsular opening. The AC-IOL was pushed further into the opening such that the lower capsular rim fit into a grooved edge of the lens. The viscoelastic material in the capsular bag was removed before capturing the optic in the opening of the capsular bag. The lens was forced downwards so that the anterior optic bag in the capsular opening and the groove of the lens was captured by the capsular rim. The AC-IOL was captured by the capsular rim along its entire circumference and the lens was fixed in place.

Once the AC-IOL was captured by the capsular opening, the empty lens capsular bag was refilled with the injectable silicone gel. This was accomplished by another insertion of a 22 gauge blunt canula behind the AC-IOL or through an incision through the cornea.

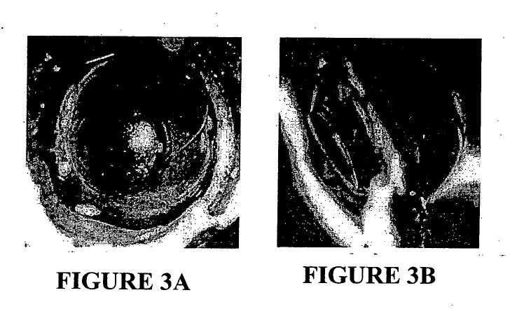

FIGS. 3A and 3B shows an excised lens from one of the pig eyes with 3A a front view and 3B a side view showing the placement of the AC-IOL within the capsular bag.

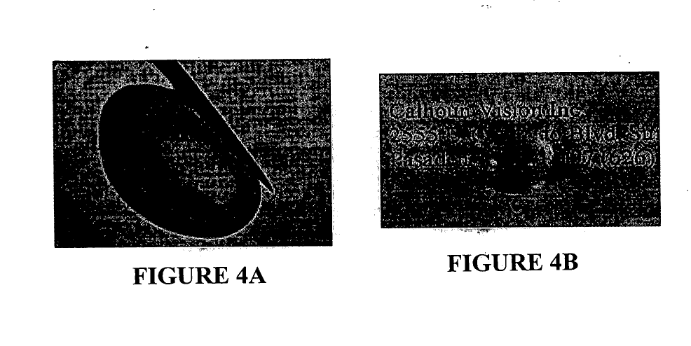

FIGS. 4A shows the feasibility of maintaining the capsular bag shape with the materials of the present invention. FIG. 4B shows the optical quality and refractive power of the capsular gel lens.

### II. Rabbit Eye Studies

Four eyes in three rabbits were successfully implanted with an AC-IOL and the capsular bag was refilled using the procedures and materials authored above. The implanted lenses and accommodation gel were allowed to remain in the rabbits for three weeks before they were sacrificed. The lenses were evaluated and corneas were removed for examination.

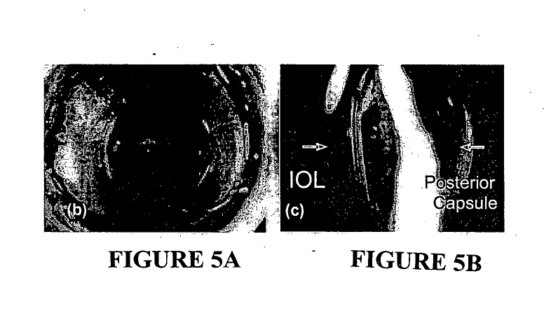

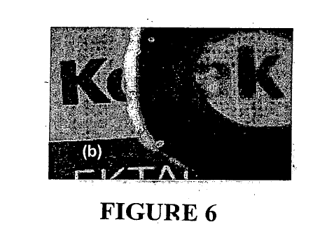

FIGS. 5 A and 5B shows a refilled capsular bag with proper placement of the AC-IOL. FIG. 6 shows the clarity of the filled capsular bag.

1. A method for providing accommodation for an intraocular lens comprising:
implanting an intraocular lens in an eye;
filling the capsular bag with an accommodation composition wherein the accommodation composition is injected and formed in situ.

2. The method of claim 1 wherein the intraocular lens is an adjustable intraocular lens.

3. The method of claim 1 wherein the accommodation composition surrounds the introcular lens.

4. The method of claim 1 wherein the introcular lens is an anterior capsule captured intraocular lens.

5. The method of claim 1 wherein said accommodation composition further comprises macromers capable of stimulus induced polymerization.

6. A method for providing accommodation for an intraocular lens comprising
implanting an intraocular lens in an eye;
filling the capsular bag with an accommodation composition, said accommodation composition comprising a polymer matrix formed in vivo; macromers dispersed throughout said polymer matrix, said macromers capable of stimulus induced polymerization;
exposing said accommodation composition to an external stimulus to induce polymerization of said macromers.

7. A method for providing accommodation for an intraocular lens comprising
implanting an intraocular lens in an eye;
forming an accommodation composition within the capsular bag of the eye said accommodation composition comprising a polymer matrix and macromers dispersed within the polymer matrix when said macromers are capable of stimulus induced polymerization.

8. A method for providing accommodation for an intraocular lens comprising forming an accommodation composition within the capsular bag of the eye said accommodation composition comprising a polymer matrix and macromers dispersed within the polymer matrix when said macromers are capable of stimulus induced polymerization.
